(19) United States
(12) Patent Application Publication
(10) Pub. No.: US 2026/0198920 A1
(43) Pub. Date: Jul. 16, 2026

(54) ANVIL BUTTRESS LOADING FOR A SURGICAL STAPLING APPARATUS

(71) Applicant: Covidien LP, Mansfield, MA (US)

(72) Inventors: Matthew J. CHOWANIEC, Madison, CT (US); Roanit A. FERNANDES, Bethany, CT (US); Jacob C. BARIL, Norwalk, CT (US); Wojciech KISIEL, North Haven, CT (US); Anthony GADDY, Windsor, CT (US); Thomas CASASANTA, JR., Southington, CT (US); Aaron J. ST SAUVEUR, Branford, CT (US)

(21) Appl. No.: 19/135,017

(22) PCT Filed: Dec. 15, 2023
(86) PCT No.: PCT/IB2023/062777

§ 371 Date: Jun. 2, 2025

Related U.S. Application Data
(60) Provisional application No. 63/433,877, filed on Dec. 20, 2022.

(51) Int. Cl. A61B 17/072; A61B 17/00; A61B 90/00

(52) U.S. Cl. CPC A61B 17/072; A61B 90/08; A61B 2017/00477; A61B 2017/07257; A61B 2017/07278; A61B 2090/0811

(57) ABSTRACT

A surgical stapling system includes an end effector and a buttress loading assembly. The end effector includes an anvil assembly. The buttress loading assembly includes an anvil buttress and an anvil buttress loader. The anvil buttress loader supports the anvil buttress and includes a support plate and a sled. The support plate includes a flexible locking arm. The sled is coupled to the support plate by the flexible locking arm. The sled supports the anvil buttress and is configured to slide the anvil buttress onto the anvil assembly. The sled is configured to move relative to the support plate when the flexible locking arm is uncoupled from the sled.

This Application claims priority from U.S. Provisional Patent Application 63/433,877, filed 20 Dec. 2022, the entire content of which is incorporated herein by reference.

### TECHNICAL FIELD

This application relates to surgical stapling systems and more particularly, to systems, devices, and methods for loading buttresses on an anvil of a surgical stapling apparatus.

### BACKGROUND

Surgical stapling apparatus are employed by surgeons to sequentially or simultaneously apply one or more rows of fasteners, e.g., staples or two-part fasteners, to body tissue for the purpose of joining segments of body tissue together. Such apparatus generally include a pair of jaws or finger-like structures between which the body tissue to be joined is placed. When the surgical stapling apparatus is actuated, or “fired,” staple drive members in one of the jaws push the surgical staples through the body tissue and into an anvil in the opposite jaw which forms the staples. If body tissue is to be removed or separated, a knife blade can be provided in one of the jaws of the apparatus to cut the body tissue between the lines of staples.

Surgical supports, e.g., meshes or buttress materials, may be used in combination with surgical stapling apparatus to bridge, repair, and/or reinforce tissue defects within a patient such as those occurring, for example, in the abdominal wall, chest wall, diaphragm, or musculo-aponeurotic areas of the body. The buttress material reinforces the staple line as well as covers the juncture of the tissues to reduce leakage prior to healing. The buttress material can help promote proper staple formation while reducing twisting/malformation caused by any misalignment of tissue and/or unusual or non-uniform tissue. The buttress material can also provide support to weakened tissue, or help address differences in the thickness of tissues.

Accordingly, buttress materials provide clinical benefits. Nonetheless, improvements are desired, for example, to reduce the complexity of manufacture and/or application of the buttress materials onto surgical stapling apparatus or into tissue, or to expand the range of application for use of the buttress materials.

### SUMMARY

There is a need for an easy, intuitive method of applying buttress material on an anvil assembly of a cartridge-based stapling system. This disclosure details such a system for loading buttress material on the anvil assembly with features that enable the buttress to remain fully loaded and retained on the anvil. Advantageously, an anvil buttress loader of a buttress loading assembly of this disclosure can act as a shipping cover for the buttress, facilitate a transfer of the buttress onto the anvil assembly, can prevent a user from deploying the buttress unless the buttress loading assembly is loaded completely onto the anvil assembly, can prevent the user from only partially loading the buttress, and can prevent the anvil buttress loader from being reset.

According to an aspect, this disclosure is directed to a buttress loading assembly for an anvil assembly of a surgical stapling apparatus. The buttress loading assembly includes an anvil buttress and an anvil buttress loader. The anvil buttress loader includes a support plate, a sled, and a shield. The support plate includes a flexible locking arm. The sled is mounted to the support plate and is selectively movable relative to the support plate in response to movement of the flexible locking arm of the support plate relative to the sled. The sled supports the anvil buttress and is configured to slide the anvil buttress onto the anvil assembly when the sled moves relative to the support plate. The shield is mounted on the sled and secured to the support plate. The shield includes a buttress finger that engages the anvil buttress and is movable relative to the shield in response to the anvil assembly being inserted into engagement with the buttress finger to enable the anvil assembly to move the flexible locking arm of the support plate relative to the sled.

In aspects, the sled may include a visual indicator protuberance, and the shield may define a visual indicator slot that receives the visual indicator protuberance of the sled. The visual indicator protuberance of the sled may slide through the visual indicator slot of the shield to indicate when the anvil buttress is loaded onto the anvil assembly. The sled may include an arch. The arch may support the visual indicator protuberance and may define a recess that is configured to receive the flexible locking arm to selectively secure the flexible locking arm to the sled. The anvil buttress may be secured to the visual indicator protuberance.

In aspects, the support plate may define an elongated lockout notch and the sled may include a lockout member that is slidably received within the elongated lockout notch. The elongated lockout notch may include a tapered surface that compresses the lockout member. The elongated lockout notch may include shoulder stops that prevent the lockout member from sliding through the elongated lockout notch when the anvil buttress is loaded on the anvil assembly.

In aspects, the support plate may include arms having teeth that are configured to secure the support plate to the anvil assembly. The sled may include cam bosses that engage the arms to uncouple the support plate from the anvil assembly when the sled is translated relative to the support plate.

In aspects, the sled may include prongs that couple to the anvil buttress to secure the anvil buttress to the sled.

According to another aspect, this disclosure is directed to a surgical stapling system. The surgical stapling system includes an end effector including an anvil assembly, and a buttress loading assembly. The buttress loading assembly includes an anvil buttress and an anvil buttress loader that supports the anvil buttress. The anvil buttress loader includes a support plate and a sled. The support plate includes a flexible locking arm. The sled is coupled to the support plate by the flexible locking arm. The sled supports the anvil buttress and is configured to slide the anvil buttress onto the anvil assembly. The sled is configured to move relative to the support plate when the flexible locking arm is uncoupled from the sled.

In aspects, the anvil buttress loader may further include a shield secured to the support plate and configured to cover the anvil buttress. The shield may include a buttress finger that engages the anvil buttress. The buttress finger may be movable relative to the anvil buttress when the anvil assembly is inserted into the shield.

In aspects, the anvil assembly may include a spring clip configured to secure a proximal end portion of the anvil buttress to the anvil assembly. The sled may include a camming rail that supports a camming shoulder. The camming shoulder may be configured to pivot the spring clip to enable the anvil buttress to extend between the spring clip and an anvil body of the anvil assembly.

In aspects, the anvil body may extend to a distal tip and the anvil buttress may define an elongated tip slot in a distal end portion of the anvil buttress. The distal tip of the anvil body may be receivable through the elongated tip slot to secure the distal end portion of the anvil buttress to the anvil assembly.

In aspects, the support plate may further include inner arms having teeth on proximal end portions of the inner arms. The anvil assembly may include wings defining windows. The teeth may be configured to be selectively pivotably received within the windows to selectively secure the support plate to the anvil assembly. The sled may include cam bosses that axially translate into engagement with the inner arms as the sled translates relative to the support plate to pivot the teeth out of the windows.

According to yet another aspect, this disclosure is directed to an anvil buttress loader. The anvil buttress loader includes a support plate, a shield, and a sled. The shield is secured to the support plate and includes a first indicator nub and a second indicator nub. The sled is mounted to the support plate. The sled is selectively movable relative to the support plate and the shield to load a buttress onto an anvil. The sled includes a pivotable arm member that selectively attaches to the anvil by a tooth supported on an end of the pivotable arm member. The sled includes a visual indicator protuberance that moves from the first indicator nub to the second indicator nub when the sled moves relative to the support plate. The sled includes a cam boss that pivots the tooth away from the anvil when the visual indictor protuberance is slid from the first indicator nub to the second indictor nub.

Other features of the disclosure will be appreciated from the following description.

### BRIEF DESCRIPTION OF THE DRAWINGS

The above and other aspects, features, and advantages of this disclosure will be apparent in light of the following detailed description when taken in conjunction with the accompanying drawings, which are incorporated in and constitute a part of this specification, wherein:

FIG. 1 is a perspective view of a surgical stapling apparatus of a surgical stapling system in accordance with the principles of this disclosure;

FIGS. 2A and 2B are perspective views of a buttress loading assembly of the surgical stapling system including the surgical stapling apparatus of FIG. 1;

FIG. 3 is perspective view, with parts separated, of the buttress loading assembly of FIGS. 2A and 2B;

FIG. 4 is a bottom, perspective view of a sled of the buttress loading assembly of FIGS. 2A and 2B;

FIG. 5 is an enlarged view of the indicated area of detail shown in FIG. 4;

FIG. 6 is an enlarged view of the indicated area of detail shown in FIG. 3;

FIG. 7 is a bottom, perspective view of a shield of the buttress loading assembly of FIGS. 2A and 2B;

FIG. 8 is an enlarged view of the indicated area of detail shown in FIG. 2A;

FIG. 9 is an enlarged, perspective view of the buttress loading assembly of FIGS. 2A and 2B with the shield and support plate of the buttress loading assembly removed for clarity;

FIG. 10 is an enlarged, cross-sectional view as taken along section line 10-10 shown in FIG. 2A;

FIG. 11 is an enlarged, cross-sectional view as taken along section line 11-11 shown in FIG. 2A;

FIGS. 12-25 are progressive views illustrating the buttress loading assembly of FIGS. 2A and 2B securing a buttress of the buttress loading assembly to an anvil of the surgical stapling apparatus of FIG. 1; and

FIG. 26 is an enlarged, perspective view showing the buttress of the buttress loading assembly of FIGS. 2A and 2B secured to the anvil of the surgical stapling apparatus of FIG. 1.

### DETAILED DESCRIPTION

Aspects of this disclosure will now be described in detail with reference to the drawing figures wherein like reference numerals identify similar or identical elements. Throughout this description, the term “proximal” refers to a portion of a structure, or component thereof, that is closer to a user, and the term “distal” refers to a portion of the structure, or component thereof, that is farther from the user. Directional reference terms, such as “top,” “bottom,” “side,” “upper,” “lower,” and the like, are used to ease description of the aspects and are not intended to have any limiting effect on the ultimate orientation of a structure or any part thereof. In addition, the term “endoscopic” is used generally to refer to endoscopic, laparoscopic, arthroscopic, and/or any other procedure conducted through a small diameter incision or cannula. Further, the term “clinician” is used generally to refer to medical personnel including doctors, nurses, surgeons, and support personnel.

In the following description, well-known functions or constructions are not described in detail to avoid obscuring this disclosure in unnecessary detail.

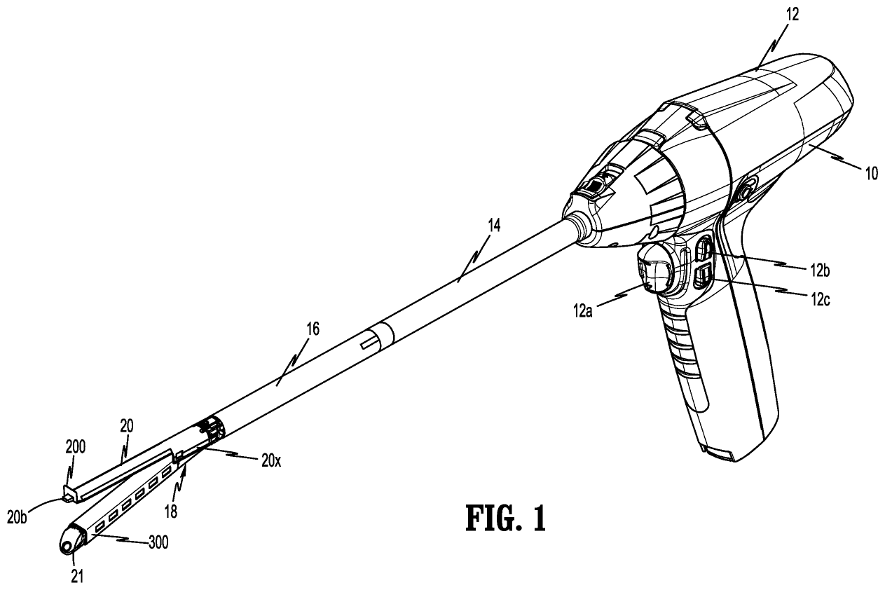

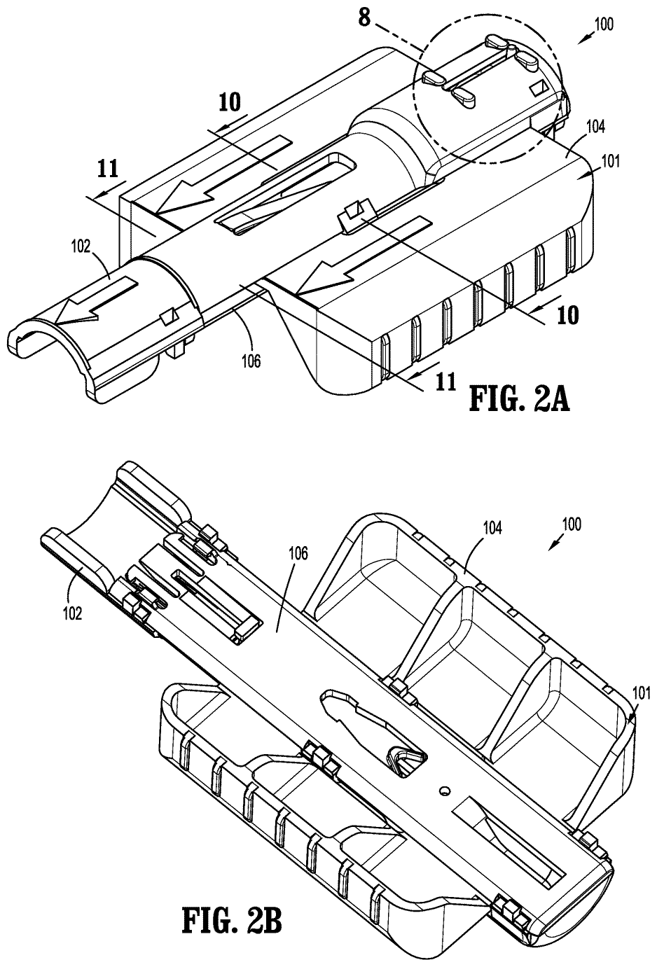

Referring now to FIGS. 1, 2A, and 2B, a surgical stapling system, in accordance with this disclosure, includes a surgical stapling apparatus or surgical stapler 10 and a buttress loading assembly 100 for use in stapling tissue and applying one or more anvil and/or cartridge buttress materials or surgical buttresses 200, 300 to the tissue. Surgical stapling apparatus 10 generally includes a handle assembly 12 and an elongated tubular body portion 14 that extends distally from handle assembly 12, which may be in the form of an adapter assembly that is selectively removable from handle assembly 12. Elongated tubular body portion 14 may include a surgical loading unit 16 that is selectively attachable to elongated tubular body portion 14. An end effector or jaw assembly 18 extends distally from elongated tubular body portion 14 (e.g., a distal end portion of surgical loading unit 16). Jaw assembly 18 includes an anvil assembly 20 and a staple cartridge assembly 21. Jaw assembly 18 may be permanently affixed to elongated tubular body portion 14 or may be detachable with respect to elongated tubular body portion 14 and thus, replaceable with a new jaw assembly 18. Anvil assembly 20 and/or staple cartridge assembly 21 is pivotable with respect to elongated tubular body portion 14 such that anvil and/or staple cartridge assemblies 20, 21 is/are movable between an open position in which anvil and staple cartridge assemblies 20, 21 are spaced apart with respect to each other (FIG. 1) and a closed position (not shown) in which anvil and staple cartridge assemblies 20, 21 are substantially adjacent each other.

Handle assembly 12 of surgical stapling apparatus 10 includes any number of actuators 12a, 12b, 12c to facilitate a firing of jaw assembly 18, an articulation and/or rotation of jaw assembly 18 relative to handle assembly 12, and/or an opening and/or closing of anvil and/or cartridge assemblies 20, 21 to clamp tissue therebetween. Jaw assembly 18 is configured to apply lines of staples (not shown) to tissue captured between anvil and staple cartridge assemblies 20, 21 when fired.

For a detailed description of the structure and function of exemplary surgical stapling apparatus, one or more components of which may be included, or modified for use with the disclosed aspects, reference may be made to U.S. Pat. Nos. 11,432,818; 8,256,656; 7,819,896; and 7,128,253, the entire contents of each of which are incorporated herein by reference. It should be appreciated that principles of this disclosure are equally applicable to surgical stapling apparatus having other configurations such as, for example, the types described in U.S. Pat. Nos., 7,334,717; 5,964,394; and 5,915,616, the entire contents of each of which are incorporated herein by reference. Accordingly, it should be understood that a variety of surgical stapling apparatus may be utilized with the surgical buttresses and/or surgical buttress applicators or loaders of this disclosure such as, for example, laparoscopic staplers, open staplers, transverse anastomosis staplers, and end-to-end anastomosis staplers having a circular staple cartridge and anvil, as well as staple cartridge assemblies housing surgical fasteners other than staples.

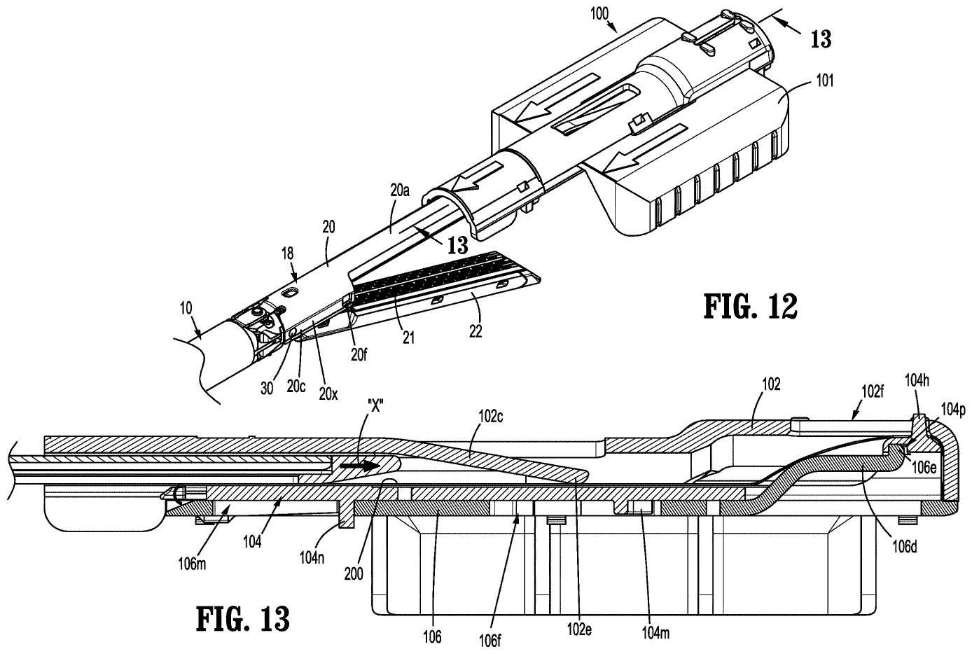

As seen in FIGS. 1, 12, and 26, anvil assembly 20 of jaw assembly 18 of surgical stapling apparatus 10 includes an anvil body 20a having a proximal end portion including a tissue stop 20x configured to prevent proximal tissue migration and a distal end portion having a distal tip 20b. Tissue stop 20x has a pair of tissue stop wings 20c, 20d that extends outwardly and downwardly from opposite side surfaces of anvil body 20a. Each wing 20c, 20d defines a pivot pin hole 20e on a proximal end portion of the respective wing for supporting pivot pins 30 and enabling pivoting movement between anvil assembly 20 and staple cartridge assembly 21. Each wing 20c, 20d further defines a window 20f in a distal end portion of the respective wing for enabling buttress loading assembly 100 to selectively secure to anvil assembly 20. Each wing 20c, 20d further includes a distal camming edge 20g. Anvil assembly 20 further includes first and second spring clips 22, 24 flexibly mounted to anvil body 20a and supported on opposite sides of a knife channel 26 defined through anvil assembly 20. First and second spring clips 22, 24 have proximal end portions 22a, 24a that are secured to anvil body 20a between wings 20c, 20d and proximal to anvil pockets 28 defined in anvil body 20a for forming staples (not shown) fired from staple cartridge assembly 21. Each of first and second spring clips 22, 24 extends distally to outer and inner split fingers 23a and 23b that are configured to flex away from anvil body 20a when a pivoting force is applied to distal end portions of first and second spring clips 22, 24, but are biased to flex back toward an unflexed position adjacent to anvil body 20a (FIG. 26) when the pivoting force is removed. Inner split fingers 23b are disposed inwardly of outer split fingers 23a adjacent the knife channel 26. Inner split finger 23b includes a proximal segment 23p and a distal segment 23d that angles away from proximal segment 23p and away from anvil body 20a. Outer split fingers 23a include a proximal segment 23pp and a distal segment 23dd that is angled (e.g., curved) toward anvil body 20a.

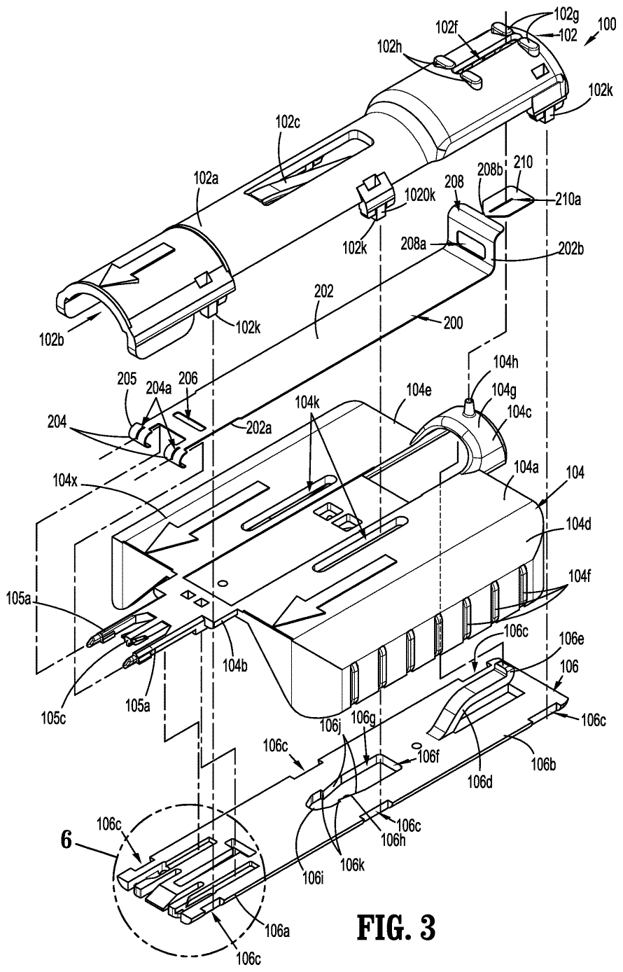

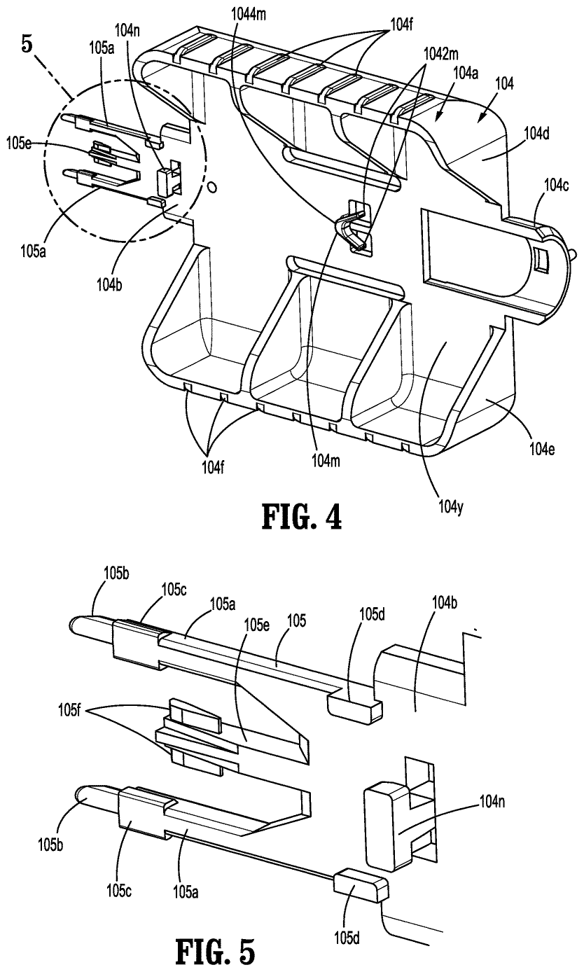

With reference to FIGS. 3-5, buttress loading assembly 100 of the disclosed surgical stapling system includes an anvil buttress 200 and an anvil buttress loader 101 for loading anvil buttress 200 onto surgical stapling apparatus 10. Advantageously, anvil buttress loader 101 of buttress loading assembly 100 acts as a shipping cover for anvil buttress 200, facilitates a transfer of the anvil buttress 200 onto the anvil assembly 20, prevents a user from deploying the anvil buttress 200 unless the buttress loading assembly 100 is inserted completely onto the anvil assembly 20, prevents a user from only partially loading the anvil buttress 200 onto the anvil assembly 20, and prevents the anvil buttress loader 101 from being reset. Indeed, anvil buttress loader 101 is a delivery system that helps to eliminate or reduce user error in the process of applying the anvil buttress 200 onto the anvil assembly 20.

Anvil buttress 200 of buttress loading assembly 100 includes a buttress body 202. Buttress body 202 has a proximal end portion 202a and a distal end portion 202b. Proximal end portion 202a of buttress body 202 includes spaced extensions 204 (e.g., laterally spaced apart from one another). Each extension 204 defines a prong opening 204a through cuffs 205 supported on a proximal end of spaced extensions 204. Cuffs 205 may have a semi-circular profile. Proximal end portion 202a of buttress body 202 further defines an elongated clip slot 206 that extends transversely across buttress body 202 and relative to a length of buttress body 202. Distal end portion 202b of buttress body 202 includes a jaw catch portion 208 that angles upwardly from buttress body 202 and defines an elongated tip slot 208a therethrough. Elongated tip slot 208a also extends transversely across buttress body 202 and relative to the length of buttress body 202. Distal end portion 202b further includes a tab 210 that extends distally from jaw catch portion 208. Tab 210 defines an indicator slot 210a that extends lengthwise along tab 210. Tab 210 is connected to jaw catch portion 208 by a frangible portion 208b to enable tab 210 to separate from buttress body 202.

Anvil buttress loader 101 of buttress loading assembly 100 includes a shield 102, a sled 104, and a support plate 106.

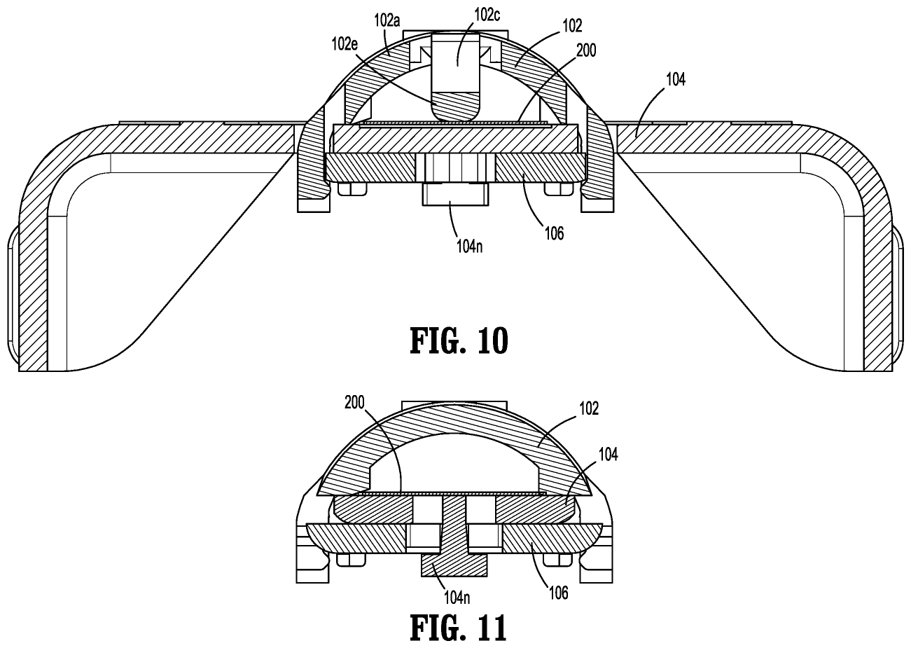

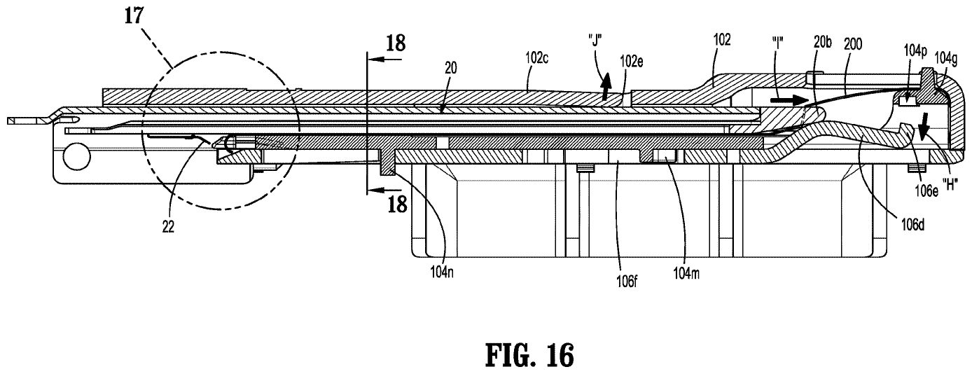

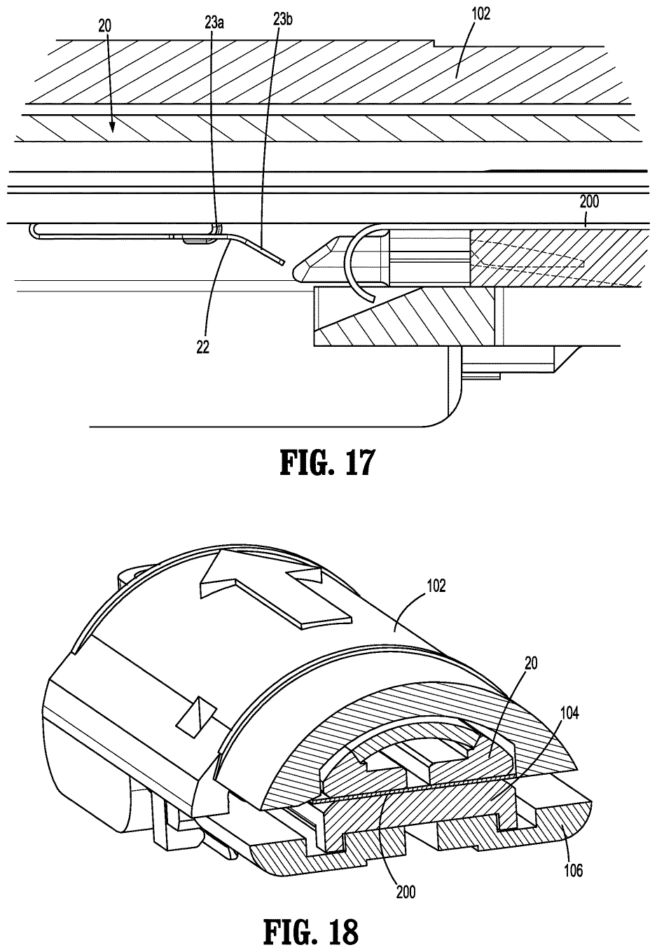

With reference to FIGS. 3, 7, 8, 13, and 25, shield 102 of anvil buttress loader 101 includes an elongated body 102a having a curved or semi-circular cross-section. Elongated body 102a defines a central channel 102b that extends along a length of elongated body 102a. Elongated body 102a includes a buttress finger 102c that depends from an intermediate portion of elongated body 102a into central channel 102b defined by elongated body 102a. Elongated body 102a further defines an elongated finger opening 102d along an intermediate portion of elongated body 102a. Buttress finger 102c has a proximal end portion secured to elongated body 102a. Buttress finger 102c extends distally downward from elongated body 102a along elongated finger opening 102d of elongated body 102a and to a free end 102e disposed in central channel 102b. Indeed, buttress finger 102c is sloped or angled relative to elongated body 102a such that free end 102e of buttress finger 102c is configured to engage a top surface of anvil buttress 200 to constrain anvil buttress 200 between shield 102 and sled 104 of anvil buttress loader 101 during shipping as seen in FIG. 10. Buttress finger 102c is flexible and configured to pivot from an initial position (FIG. 13) to a flexed position (FIG. 16, as indicated by arrow “J” in response an axial force from distal tip 20b of anvil assembly 20 as anvil assembly 20 is moved (e.g., axially translated) into engagement with buttress finger 102c, as indicated by arrow “X.” Elongated body 102a further defines a visual indicator slot 102f that extends lengthwise along a distal end portion of elongated body 102a. Elongated body 102a also includes distal indicator nubs 102g adjacent a distal end of visual indicator slot 102f and proximal indicator nubs 102h adjacent a proximal end of visual indicator slot 102f. Distal indicator nubs 102g may have a surface that is colored a first color (e.g., red) and proximal indicator nubs 102h may have surface that is colored a second color (e.g., green) so that proximal and distal indicator nubs 102g, 102h can function as position indicators of sled 104 relative to shield 102 and/or buttress loading state (e.g., buttress 200 loaded and/or unloaded onto anvil assembly 20). Elongated body 102a further includes mounting hooks 102k at spaced apart locations along both sides of elongated body 102a for securing shield 102 to support plate 106.

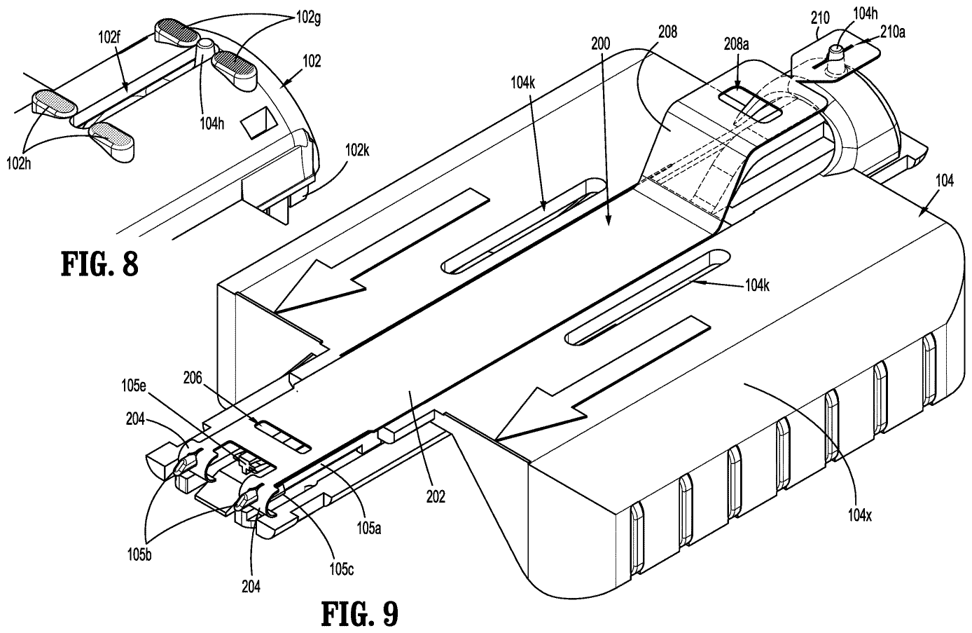

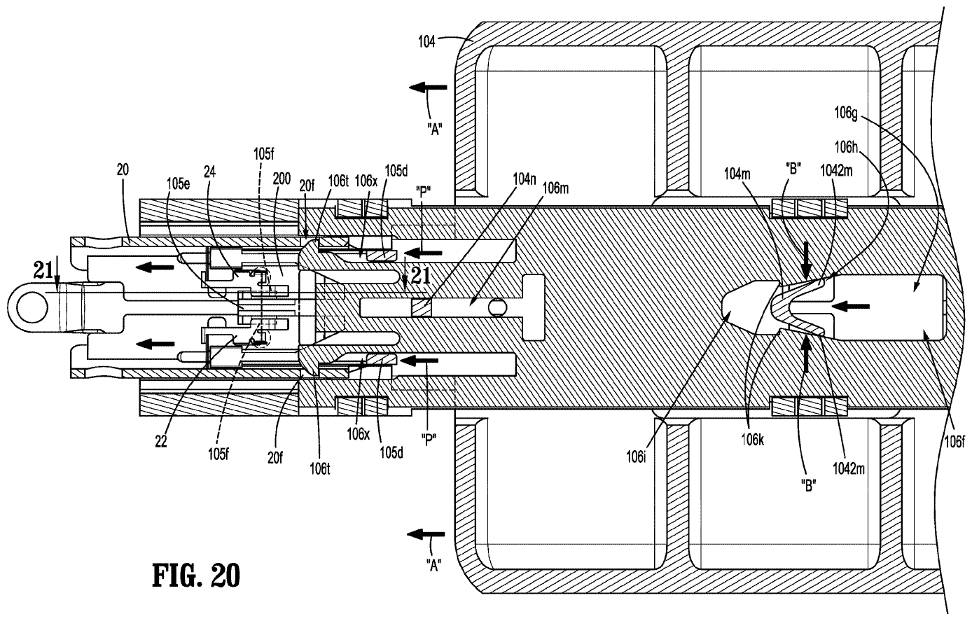

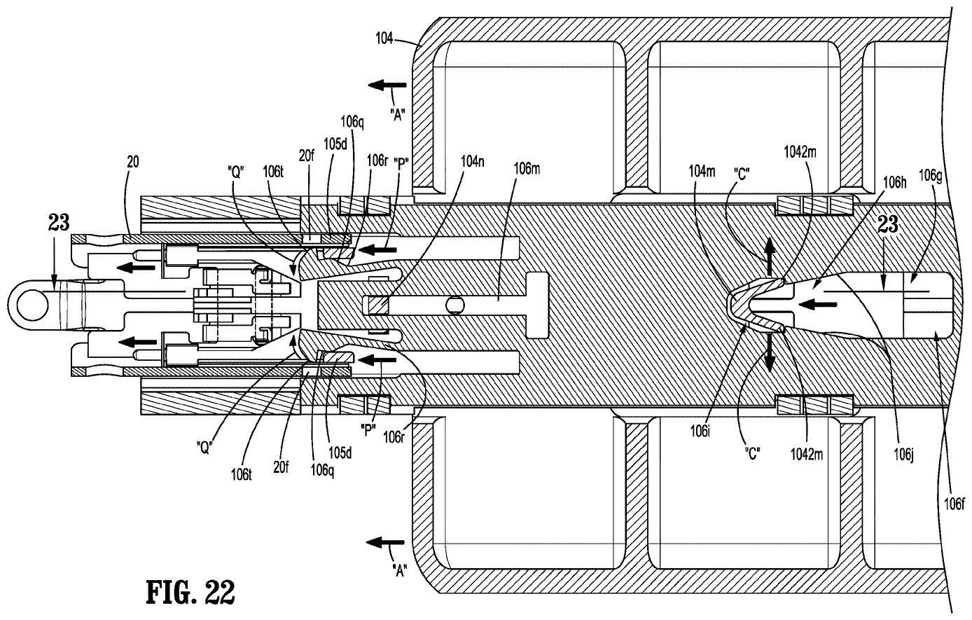

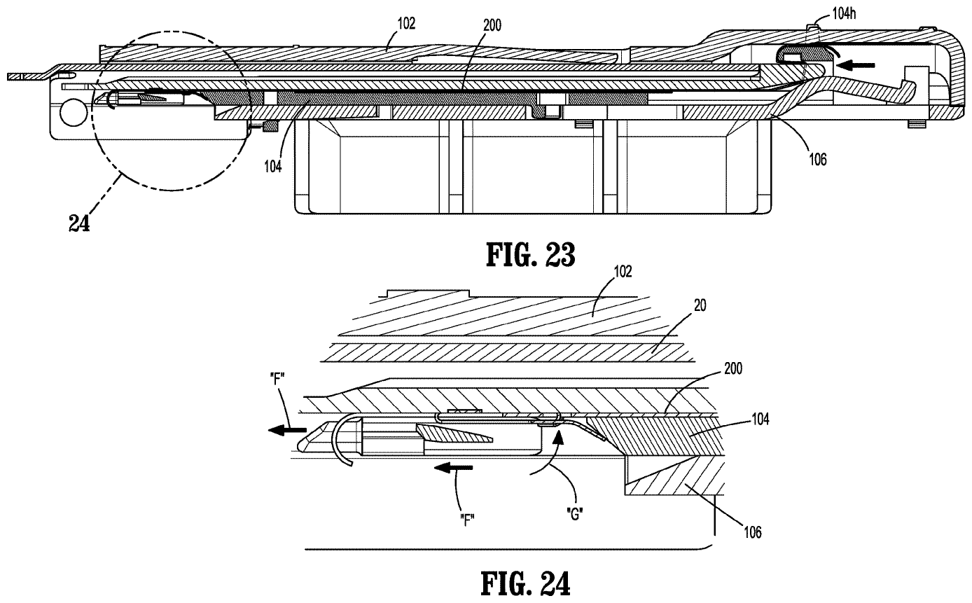

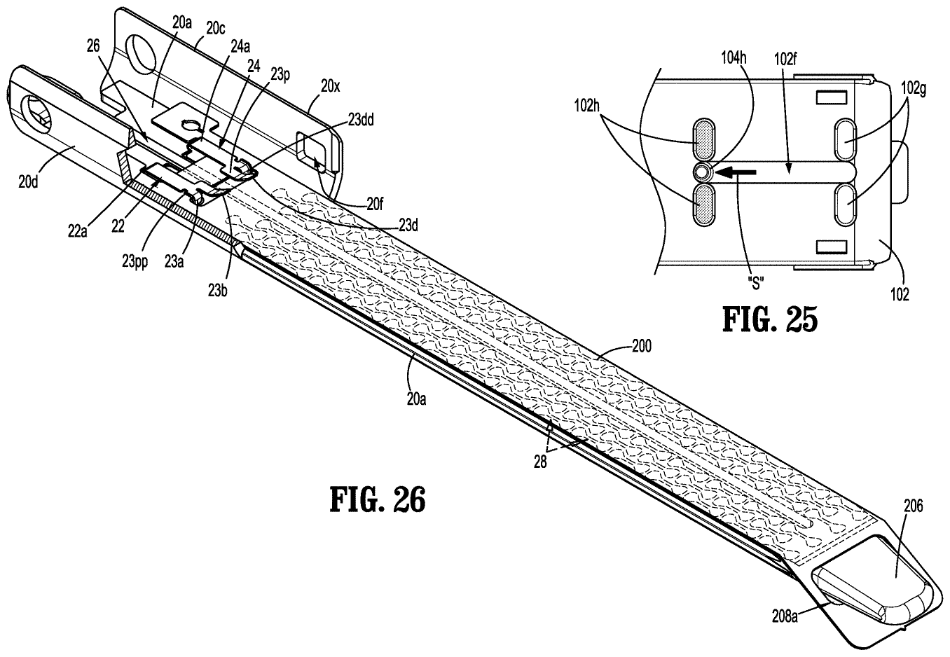

Referring now to FIGS. 3-5, 8, 9, 13, 16, 20 and 22, sled 104 of anvil buttress loader 101 includes a sled body 104a, which is shown as rectangular although other shapes are envisioned. Sled body 104a includes a proximal end portion 104b and a distal end portion 104c. Sled body 104a includes a first wing 104d and a second wing 104e that extend in opposite directions to opposite sides of sled body 104a. The sides of sled body 104a extend between a top surface 104x and a bottom surface 104y of sled body 104a. Each of first and second wings 104d, 104e has a plurality of spaced ribs 104b that extends along the respective sides of sled body 104a to facilitate user gripping of sled body 104a. Sled body 104a further includes an arch 104g extending over top surface 104x of sled body 104a at distal end portion 104c of sled body 104a. Arch 104g further includes a visual indicator protuberance 104h that projects upwardly from an apex of a top surface of arch 104g and is slidably received within visual indicator slot 102f of shield 102, as seen in FIGS. 8 and 25, and as indicted by arrow “S.” Visual indicator protuberance 104h is also received within indicator slot 210a of tab 210 of anvil buttress 200, as seen in FIG. 9, to enable tab 210 of anvil buttress 200 to extend over arch 104g to maintain jaw catch portion 208 and elongated tip slot 208a of jaw catch portion 208 elevated above buttress body 202 at the distal end portion of anvil buttress 200 for facilitating receipt of distal tip 20b of anvil body 20a through elongated tip slot 208a. Arch 104g further defines a tab recess 104p (see FIGS. 13 and 16) in bottom surface of arch 104g adjacent to the apex of arch 104g. Sled body 104a further defines elongated slide channels 104k that are aligned with intermediate mounting hooks 1020k (see FIG. 3) of mounting hooks 102k of shield 102 for slidably receiving intermediate mounting hooks 1020k of shield 102 within elongated slide channels 104k of sled 104 to enable sled 104 to slide relative to shield and support plate assemblies 102, 106, as indicated by arrows “A” in FIGS. 20 and 22.

Bottom surface 104y of sled body 104a includes a lockout member 104m depending from an intermediate portion of bottom surface 104y. Lockout member 104m is shown having a chevron shape, although any suitable shape is envisioned. Lockout member 104m includes legs 1042m that are connected at a rounded tip 1044m pointing in the proximal direction. Legs 1042m are configured to flex inwardly toward one another, as indicated by arrows “B” (see FIG. 20) upon application of a compression force, and configured to return to their initial position (e.g., unflexed) upon removal of the compression force, as indicated by arrows “C” (see FIG. 22). Proximal to lockout member 104m is a guide member 104n having a T-shaped configuration, although any suitable shape is envisioned, that depends from bottom surface 104y of sled body 104a adjacent proximal end portion 104b of sled body 104a.

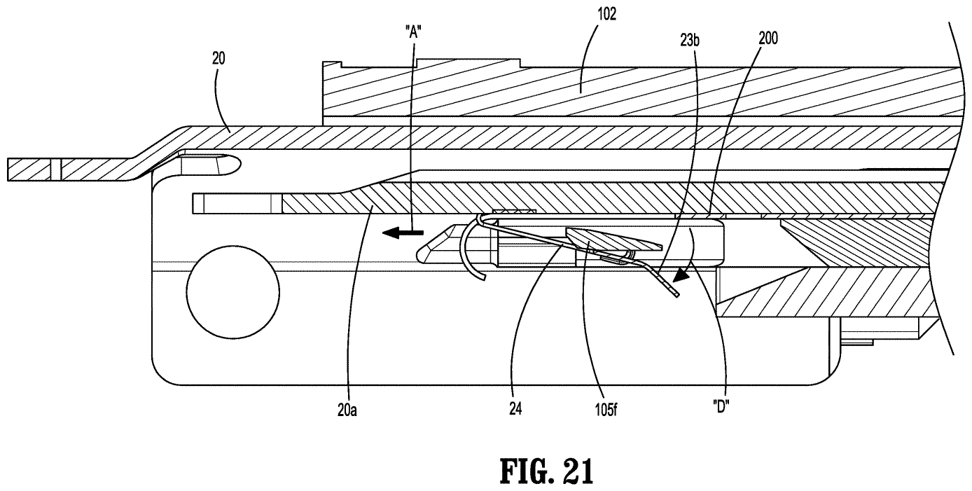

With reference also to FIGS. 3, 5, 9, 21, and 24, sled body 104a of sled 104 further includes a head assembly 105 extending proximally from proximal end portion 104b of sled body 104a. Head assembly 105 includes spaced prongs 105a having a nose 105b that is received through prong opening 204a of extensions 204 of anvil buttress 200. Spaced prongs 105a further include abutments 105c distal to nose 105b that limit distal sliding movement of extensions 204 along prongs 105a to maintain anvil buttress 200 extended along anvil buttress loader 101 as shown in FIG. 9. Head assembly 105 further includes cam bosses 105d adjacent a distal end portion of prongs 105a and depending from bottom surface 104y of sled body 104a. Head assembly 105 further includes a camming rail 105e extending proximal from sled body 104a and disposed between spaced prongs 105a. Caming rail 105e includes camming shoulders 105f disposed on opposed sides of a proximal end portion of camming rail 105e. Camming shoulders 105f are configured to cam along inner split fingers 23b of first and second spring clips 22, 24 to flex or pivot first and second spring clips 22, 24 downwardly away from anvil body 20a, as indicated by arrow “D,” as sled 104a slides proximally relative to shield 102, as indicated by arrows “A” (see FIG. 21). Camming shoulders 105f are also configured to enable first and second spring clips 22, 24 to cam proximally beyond inner split fingers 23b of first and second spring clips 22, 24, as indicated by arrows “F” (FIG. 24) so that first and second spring clips 22, 24 can bias or flex back to their initial positions relative to anvil body 20a, as indicated by arrow “G” (e.g., in close approximation to anvil body 20a) such that outer split fingers 23a of first and second spring clips 22, 24 can be received within elongated clip slot 206 of anvil buttress 200 for securing the proximal end portion of anvil buttress 200 to anvil body 20a.

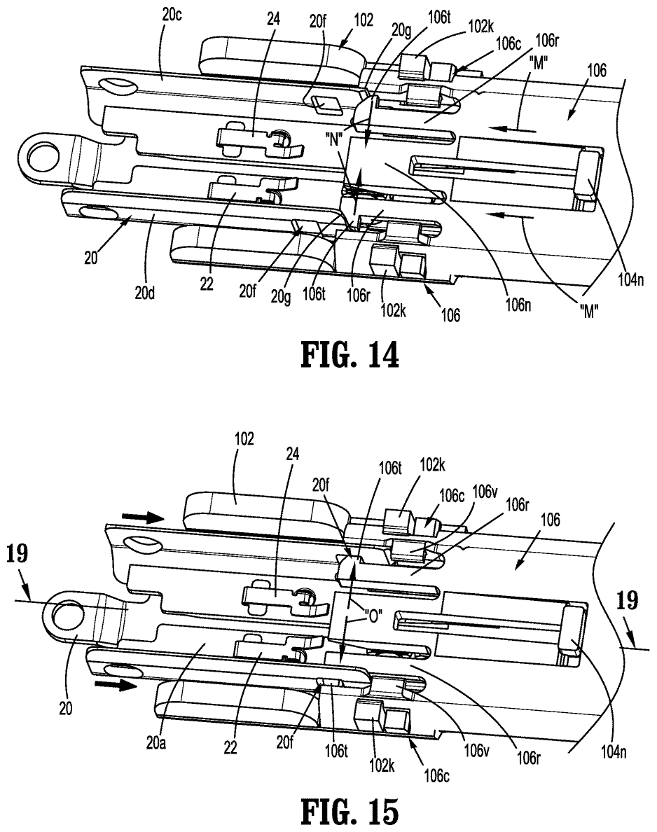

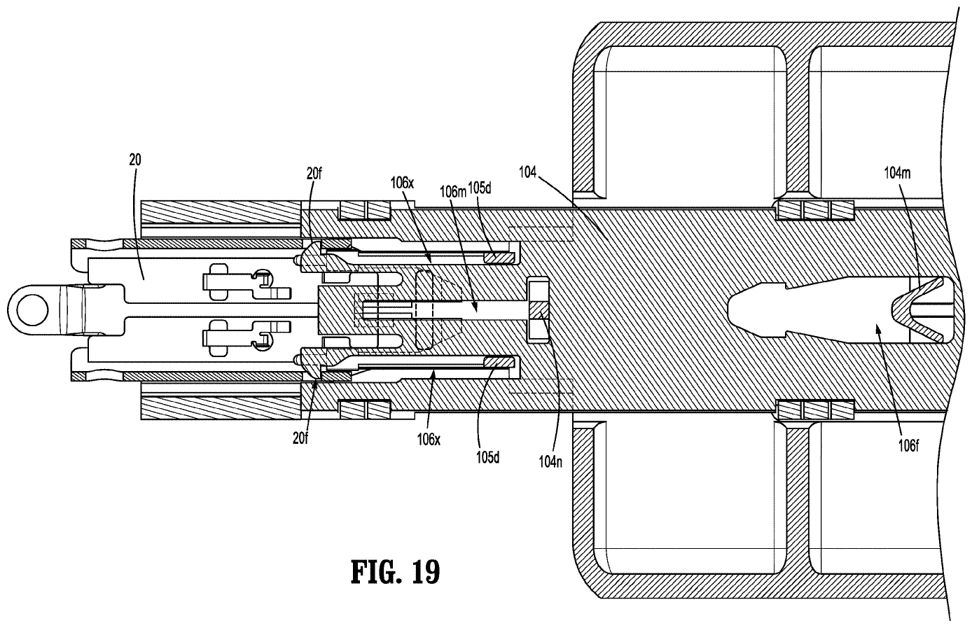

Turning now to FIGS. 3, 6, 11, 14, 15, and 19, support plate 106 of anvil buttress loader 101 includes a proximal end portion 106a and a distal end portion 106b. Support plate 106 defines mounting recesses 106c defined at spaced locations along side surfaces of support plate 106 which are aligned with, and configured to engage with, mounting hooks 102k of shield 102 to enable shield 102 to snap-fit onto support plate 106 for fixedly securing shield 102 and support plate 106 together as seen in FIGS. 14 and 15. Support plate 106 further includes a flexible locking arm 106d, which may have a curvilinear configuration, disposed at distal end portion 106b and extending upwardly from support plate 106. Flexible locking arm 106d is configured to flex or pivot downwardly, as indicated by arrow “H” in response to an axial force “I” imparted from distal advancement of distal tip 20b of anvil assembly 20 into flexible locking arm 106d, as seen in FIG. 16. Locking arm 106d further includes a locking tab 106e extending upwardly from a distal end of locking arm 106d that is selectively receivable within tab recess 104p of sled 104 to secure sled 104 to support plate 106 and prevent sled 104 from longitudinally translating relative to support plate 106, preventing a user from deploying the anvil buttress 200 unless the anvil buttress loader 101 is inserted completely onto the anvil assembly 20.

As best seen in FIG. 3, support plate 106 of anvil buttress loader 101 further defines an elongated lockout notch 106f in an intermediate portion of support plate 106 that slidably supports lockout member 104m of sled 104. Elongated lockout notch 106f includes a distal portion 106g, an intermediate portion 106h, and a proximal portion 106i. In the distal portion 106g, lockout member 104m of sled is configured to slid freely along support plate 106. Intermediate portion 106h and proximal portion 106i are separated by a shoulder stops 106k. In the intermediate portion 106h, legs 1042m of lockout member 104m are compressed together by tapered surface 106j, as indicated by arrows “B” and seen in FIG. 20, to urge lockout member 104m into proximal portion 106i where legs 1042m bias outwardly, as indicated by arrows “C”, such that shoulder stops 106k prevent lockout member 104m from sliding back into intermediate or distal portions 106h, 106g as seen in FIG. 22. When lockout member 104m is prevented from sliding in the distal direction, sled 104 cannot move relative to support plate 106, preventing anvil buttress loader 101 from being reset.

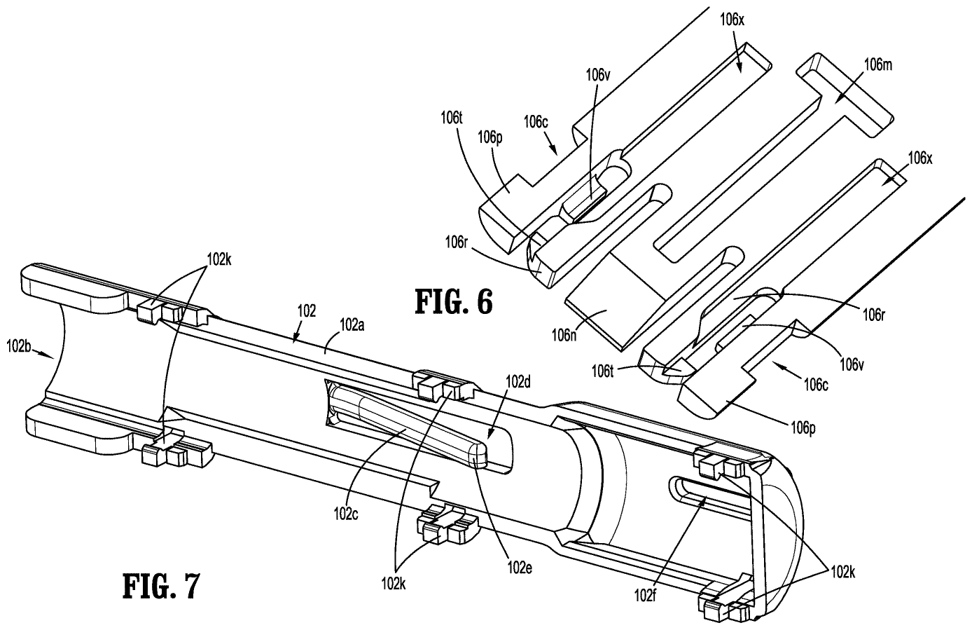

With reference now to FIG. 6, support plate 106 further defines a T-shaped slot 106m in the proximal end portion of support plate 106 that slidably supports guide member 104n of sled 104 to guide sled 104 along support plate 106. The proximal end portion of support plate 106 includes a central finger 106n that extends proximally from support plate 106 and into which a proximal portion of T-shaped slot 106m extends. The proximal end portion of support plate 106 further includes outer arms 106p and inner arms 106r that are flexibly movable relative to outer arms 106p and central finger 106n. Each of inner arms 106r has a tooth 106t that extends outwardly and away from central finger 106n and toward a respective one of outer arms 106p. Each of inner arms 106r further includes an elbow 106q that is configured to engage cam bosses 105d of sled 104. Elbows 106q may have a curvilinear configuration. Outer arms 106p further include anvil stops 106v that limit movement of anvil assembly 20 relative to support plate 106 (see FIG. 15). The proximal end portion of support plate 106 also defines spaced cam boss channels 106x configured to slidably receive cam bosses 105d of sled 104 as sled 104 slides relative to support plate 106.

With reference to FIGS. 14, 15, and 19, to prevent the user from only partially loading the anvil buttress 200, anvil assembly 20 and anvil buttress loader 101 are approximated (e.g., longitudinal translation as indicted by arrows “M”) such that distal camming edge 20g of wings 20c, 20d of anvil assembly 20 engage teeth 106t of support plate 106, causing teeth 106t and inner arms 106r to flex or pivot inwardly toward central finger 106n, as indicated by arrows “N” so that teeth 106t can along an inner surface of wings 20c, 20d as anvil buttress loader 101 longitudinally translates toward anvil assembly 20 until teeth 106t align with windows 20f of wings 20c, 20d for enabling teeth 106t to snap into windows 20f for locking support plate 106 to anvil assembly 20, as indicted by arrows “O.” In this position, as seen in FIG. 15, anvil stops 106v abut against the distal end of wings 20c, 20d of anvil assembly 20 and prevent further insertion of anvil assembly 20 relative to support plate 106.

To disengage teeth 106t of support plate 106 from windows 20f of anvil assembly 20, sled 104 is advanced toward anvil assembly 20 and relative to support plate 106 such that cam bosses 105d of sled slide along cam boss channels 106x of support plate 106, as indicated by arrows “P” (FIG. 20). As cam bosses 105d and sled 104 continue to slide in a proximal direction relative to support plate 106, anvil buttress 200 becomes fully loaded onto anvil assembly 20 and cam bosses 105d engage elbows 106q of inner arms 106r to cause teeth 106t and inner arms 106r to pivot out of windows 20f of anvil assembly 20, as indicated by arrows “Q” shown in FIG. 22. In this position, visual indicator protuberance 104h will be disposed adjacent proximal indicator nubs 102h, as seen in FIG. 25, to provide an indication to the clinician that lockout member 104m is locked within proximal portion 106i of elongated lockout notch 106f to prevent movement of sled 104 relative to support plate 106, and that anvil buttress 200 is fully loaded on anvil assembly 20. In particular, as seen in FIG. 26, when anvil buttress 200 is fully loaded on anvil assembly 20, distal tip 20b of anvil assembly 20 is secured within elongated tip slot 208a at distal end portion 202b of anvil buttress 200 and the proximal end portion 202a of anvil buttress 200 is secured between first and second clips 22, 24 and anvil body 20a. With teeth 106t pivoted out of windows 20f, anvil buttress loader 101 can be separated from anvil buttress 200 and anvil assembly 20 by distal removal of anvil buttress loader 101.

The buttresses of this disclosure may be fabricated from biocompatible materials which are bioabsorbable or non-absorbable, natural materials, or synthetic materials. It should be understood that any combination of natural, synthetic, bioabsorbable, and/or non-bioabsorbable materials may be used to form the surgical buttresses. The buttresses may be biodegradable (e.g., formed from bioabsorbable and bioresorbable materials) such that the buttresses decompose or are broken down (physically or chemically) under physiological conditions in the body, and the degradation products are excretable or absorbable by the body. Components or portions of the buttresses may be formed from the same material or different materials.

In aspects, at least a portion of the buttresses are made from biodegradable materials selected from the following group: natural collagenous materials, cat gut, and synthetic resins including those derived from alkylene carbonates, trimethylene carbonate, tetramethylene carbonate, caprolactone, valerolactone, dioxanone, polyanhydrides, polyesters, polyacrylates, polymethylmethacrylates, polyurethanes, glycolic acid, lactic acid, glycolide, lactide, polyhydroxy butyrates, polyorthoester, polyhydroxy alkanoates, homopolymers thereof, and copolymers thereof. In aspects, at least a portion of the surgical buttresses may be made from non-biodegradable materials selected from the following group: polyolefins, polyethylene, polydimethylsiloxane, polypropylene, copolymers of polyethylene and polypropylene, blends of polyethylene and polypropylene, ultra-high molecular weight polyethylene, polyamides, polyesters, polyethylene terephthalate, polytetrafluoroethylene, polyether-esters, polybutester, polytetramethylene ether glycol, 1,4-butanediol, and polyurethanes.

The buttresses may be porous, non-porous, or combinations thereof. Suitable porous structures include, for example, fibrous structures (e.g., knitted structures, woven structures, and non-woven structures) and/or foams (e.g., open or closed cell foams). Suitable non-porous structures include, for example, films. The buttresses, or portions thereof, may be a non-woven structure formed by melt-blown or melt-spun methods, a mesh material, a braid material, and/or a molded or extruded sheet. The buttresses, or portions thereof, may be a single porous or non-porous layer, or include a plurality of layers including any combination of porous and/or non-porous layers.

The buttresses may be provided and/or sold as part of the buttress loader and/or loading unit. Alternatively, the buttress(es), the buttress loader, and/or the buttress loading assemblies or units may be provided and/or sold separately and assembled by the user. In aspects, one or more buttresses, one or more buttress loaders, and/or loading assemblies are provided in a kit. In some aspects, the kit further includes one or more end effectors (and/or surgical loading units) and, in certain aspects, the kit further includes a surgical stapler.

In any of the aspects disclosed herein, the buttresses can include, or be used with, brachytherapy, chemotherapy, other medical materials, or pharmaceuticals. The buttress can have pockets, apertures, or other features for retaining brachytherapy seeds with the buttress, or brachytherapy seeds or materials can be incorporated into a suture or sutures that are threaded into or through the buttress or otherwise attached thereto. A coating having brachytherapy materials can be applied to the buttress by spraying or dipping. Chemotherapy pharmaceuticals or agents can be incorporated into the buttress, coated thereon, or otherwise applied as part of a suture or other feature secured to the buttress.

As can be appreciated, securement of any of the components of the presently disclosed apparatus can be effectuated using known securement techniques such welding, crimping, gluing, fastening, etc.

The various aspects disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that the present disclosure is not limited to the precise aspects described, and that various other changes and modifications may be effected by one skilled in the art without departing from the scope or spirit of the disclosure. Additionally, it is envisioned that the elements and features illustrated or described in connection with one exemplary aspect may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

What is claimed is:

1. A buttress loading assembly for an anvil assembly of a surgical stapling apparatus, the buttress loading assembly comprising:
an anvil buttress; and
an anvil buttress loader including:
a support plate including a flexible locking arm;
a sled mounted to the support plate and selectively movable relative to the support plate in response to movement of the flexible locking arm of the support plate relative to the sled, the sled supporting the anvil buttress and configured to slide the anvil buttress onto the anvil assembly when the sled moves relative to the support plate; and
a shield mounted on the sled and secured to the support plate, the shield including a buttress finger that engages the anvil buttress and is movable relative to the shield in response to the anvil assembly being inserted into engagement with the buttress finger to enable the anvil assembly to move the flexible locking arm of the support plate relative to the sled.

2. The buttress loading assembly of claim 1, wherein the sled includes a visual indicator protuberance, and the shield defines a visual indicator slot that receives the visual indicator protuberance of the sled, wherein the visual indicator protuberance of the sled slides through the visual indicator slot of the shield to indicate when the anvil buttress is loaded onto the anvil assembly.

3. The buttress loading assembly of claim 2, wherein the sled includes an arch, the arch supporting the visual indicator protuberance and defining a recess that is configured to receive the flexible locking arm to selectively secure the flexible locking arm to the sled.

4. The buttress loading assembly of claim 2, wherein the anvil buttress is secured to the visual indicator protuberance.

5. The buttress loading assembly of claim 1, wherein the support plate defines an elongated lockout notch, and the sled includes a lockout member that is slidably received within the elongated lockout notch.

6. The buttress loading assembly of claim 5, wherein the elongated lockout notch includes a tapered surface that compresses the lockout member.

7. The buttress loading assembly of claim 5, wherein the elongated lockout notch includes shoulder stops that prevent the lockout member from sliding through the elongated lockout notch when the anvil buttress is loaded onto the anvil assembly.

8. The buttress loading assembly of claim 1, wherein the support plate includes arms having teeth that are configured to secure the support plate to the anvil assembly.

9. The buttress loading assembly of claim 8, wherein the sled includes cam bosses that engage the arms to uncouple the support plate from the anvil assembly when the sled is translated relative to the support plate.

10. The buttress loading assembly of claim 1, wherein the sled includes prongs that couple to the anvil buttress to secure the anvil buttress to the sled.

11. A surgical stapling system, comprising:
an end effector including an anvil assembly; and
a buttress loading assembly comprising:
an anvil buttress; and
an anvil buttress loader that supports the anvil buttress, the anvil buttress loader including:
a support plate including a flexible locking arm; and
a sled coupled to the support plate by the flexible locking arm, the sled supporting the anvil buttress and configured to slide the anvil buttress onto the anvil assembly, the sled configured to move relative to the support plate when the flexible locking arm is uncoupled from the sled.

12. The surgical stapling system of claim 11, wherein the anvil buttress loader further includes a shield secured to the support plate and configured to cover the anvil buttress.

13. The surgical stapling system of claim 12, wherein the shield includes a buttress finger that engages the anvil buttress.

14. The surgical stapling system of claim 13, wherein the buttress finger is movable relative to the anvil buttress when the anvil assembly is inserted into the shield.

15. The surgical stapling system of claim 11, wherein the anvil assembly includes a spring clip configured to secure a proximal end portion of the anvil buttress to the anvil assembly.

16. The surgical stapling system of claim 15, wherein the sled includes a camming rail that supports a camming shoulder, the camming shoulder configured to pivot the spring clip to enable the anvil buttress to extend between the spring clip and an anvil body of the anvil assembly.

17. The surgical stapling system of claim 11, wherein the anvil body extends to a distal tip and wherein the anvil buttress defines an elongated tip slot in a distal end portion of the anvil buttress, the distal tip of the anvil body receivable through the elongated tip slot to secure the distal end portion of the anvil buttress to the anvil assembly.

18. The surgical stapling system of claim 11, wherein the support plate includes inner arms having teeth on proximal end portions of the inner arms, and wherein the anvil assembly includes wings defining windows, the teeth configured to be selectively pivotably received within the windows to selectively secure the support plate to the anvil assembly.

19. The surgical stapling system of claim 18, wherein the sled includes cam bosses that axially translate into engagement with the inner arms as the sled translates relative to the support plate to pivot the teeth out of the windows.

20. An anvil buttress loader, comprising:
a support plate;
a shield secured to the support plate and including first and second indicator nubs; and
a sled mounted to the support plate, the sled selectively movable relative to the support plate and the shield to load a buttress onto an anvil, the sled including a pivotable arm member that selectively attaches to the anvil by a tooth supported on an end of the pivotable arm member, the sled including a visual indicator protuberance that moves from the first indicator nub to the second indicator nub when the sled moves relative to the support plate, the sled including a cam boss that pivots the tooth away from the anvil when the visual indictor protuberance is slid from the first indicator nub to the second indictor nub.
